(19) United States
(12) Patent Application Publication
(10) Pub. No.: US 2007/0023694 A1
(43) Pub. Date: Feb. 1, 2007

(54) Method of removing particles on an object, apparatus for performing the removing method, method of measuring particles on an object and apparatus for performing the measuring method

(75) Inventors: Sang-Yup Kim, Gyeonggi-do (KR); Sung-Soo Jang, Gyeonggi-do (KR); Guk-Pil Kim, Gyeonggi-do (KR); Tae-Jung Kim, Seoul (KR)

Correspondence Address: FILDES & OUTLAND, P.C., 20916 MACK AVENUE, SUITE 2, GROSSE POINTE WOODS, MI, 48236, US

(73) Assignee: Komico Ltd.

(21) Appl. No.: 11/541,223

(22) Filed: Sep. 29, 2006

Related U.S. Application Data
(63) Continuation of application No. 11/481,340, filed on Jul. 5, 2006.

(30) Foreign Application Priority Data
Jul. 10, 2005 (KR) P2005-94084

(51) Int. Cl. A61N 5/00

(52) U.S. Cl. 250/492.2

(57) ABSTRACT

In a method of removing particles on an object, a fluid is injected into a spacer where the object is placed to remove foreign substances in the space. A first light is irradiated to the object to remove charges between an upper face of the object and the particles, and inner walls of holes and the particles. A second light is irradiated to the object to remove moisture droplets between the upper face of the object and the particles, and the inner walls of the holes and the particles. A third light is irradiated to the object to remove static electricity between the upper face of the object and the particles, and the inner walls of the holes and the particles. A fluid is applied to the upper face and the holes of the object to remove the particles from the object.

### CROSS-REFERENCE TO RELATED APPLICATIONS

This application is a continuation-in-part of U.S. patent application Ser. No. 11/481,340, entitled “METHOD OF REMOVING PARTICLES ON AN OBJECT, APPARATUS FOR PERFORMING THE REMOVING METHOD, METHOD OF MEASURING PARTICLES ON AN OBJECT AND APPARATUS FOR PERFORMING THE MEASURING METHOD” and filed on Jul. 5, 2006. This application claims priority under 35 USC § 119 to Korean Patent Application No. 2005-94084 filed on Oct. 7, 2005, the contents of which are herein incorporated by reference in their entirety.

### BACKGROUND OF THE INVENTION

1. Field of the Invention

Example embodiments of the present invention relate to a method and an apparatus for removing particles on an object, and a method and an apparatus for measuring particles on an object using the same. More particularly, example embodiments of the present invention relate to a method of removing particles on inner walls of a hole in an object, such as a substrate for a semiconductor device or a flat display device, an apparatus for performing the removing method, a method of measuring the number of particles using the removing method, and an apparatus for performing the measuring method.

2. Description of the Related Art

Recently, as semiconductor devices and flat display devices have become highly integrated, contaminants such as particles, which have adverse effects on the operation of the semiconductor devices and the flat display devices, have been strictly managed. Therefore, methods of effectively removing particles on a substrate for a semiconductor device or a flat display device have been proposed. Further, in order to check the efficiency of the particle removal, methods of measuring the number of particles have been proposed.

A detector for detecting particles on an object is disclosed in Korean Patent Laid-Open Publication No. 2003-34179. The detector includes a scanner having at least one opening, a particle counter for counting the number of particles that pass through the opening of the scanner, a pump for sucking the particles into the particle counter, and a controller for controlling a speed of the pump.

However, as a particle to be removed from an object has a diameter of no more than about 0.1 μm, the particles may not be effectively removed from the object using the conventional method, because a strong adhesion force between the particle having the diameter of no more than about 0.1 μm and a surface of the object exists.

Specifically, the strong adhesion force, such as a charge force caused by charges charged on the surface of the object, a capillary force caused by fine moisture droplets between the surface of the object and the particles, or an electrostatic force caused by static electricity formed between the surface of the object and the particles, exists between the minute particles and the object. Since the above-mentioned strong adhesion force exists between the minute particles and the object, the minute particles may not be readily removed from the object using the conventional method. As a result, the minute particles remain on the substrate of the semiconductor device or the flat display device so that the semiconductor device or the flat display device may malfunction due to the remaining particles.

Particularly, when the substrate for the semiconductor substrate or the flat display device has a hole, the particles may be stuck on inner walls of the hole. The particles on the inner walls of the hole may not be readily removed from the substrate.

### SUMMARY OF THE INVENTION

Example embodiments of the present invention provide a method of removing particles on inner walls of an object that is capable of readily removing the particles from the object.

Example embodiments of the present invention also provide an apparatus for performing the above-mentioned removing method.

Example embodiments of the present invention still also provide a method of measuring particles on an object using the above-mentioned removing method.

Example embodiments of the present invention yet still also provide an apparatus for performing the above-mentioned measuring method.

In a method of removing particles on an object in accordance with one aspect of the present invention, an adhesion force between the particles and the object is removed using a light. The particles are then removed from the object using a fluid.

According to one example embodiment, the particles may be stuck on inner walls of a hole formed through the object. The fluid may be introduced into the hole to remove the particles from the hole.

According to another example embodiment, removing the adhesion force may include irradiating the light to the object.

According to still another example embodiment, the light is irradiated to the fluid to remove the adhesion force and simultaneously remove the particles.

In a method of measuring particles on an object in accordance with another aspect of the present invention, an adhesion force between the particles and the object is removed using a light. The particles are then removed from the object using a fluid. The number of the removed particles is counted.

According to one example embodiment, the particles may be stuck on inner walls of a hole formed through the object. The fluid may be introduced into the hole to remove the particles from the hole.

According to another example embodiment, counting the number of the removed particles may include measuring a first flow rate of a first fluid applied to the object, measuring a second flow rate of a second fluid used for counting the particles, counting the number of particles in the second fluid, calculating a surface area of the object, obtaining a value by multiplying a flow rate ratio of the first flow rate with respect to the second flow rate by the number of the particles in the second fluid, and dividing the value by the surface area of the object to obtain particles by unit areas of the object.

An apparatus for removing particles on an object in accordance with still another aspect of the present invention includes a light-irradiating unit for irradiating a light onto the object to remove an adhesion force between the object and the particles. A fluid-supplying unit removes the particles from the object using a fluid.

According to one example embodiment, the light-irradiating unit may irradiate the light to the fluid to remove the adhesion force and simultaneously remove the particles.

An apparatus for measuring particles on an object in accordance with still another aspect of the present invention includes a light-irradiating unit for irradiating a light onto the object to remove an adhesion force between the object and the particles. A fluid-supplying unit removes the particles from the object using a fluid. A counting unit counts the number of the removed particles. A suction unit sucks the removed particles into the counting unit.

According to one example embodiment, a discharge unit may discharge a fluid that is sucked into the counting unit by the suction unit.

According to still another example embodiment, a fluid-returning unit may be arranged between the discharge unit and the fluid-supplying unit. The fluid-returning unit returns the discharged fluid to the fluid-supplying unit to recycle the discharge fluid by the fluid-returning unit.

According to the present invention, the adhesion force, such as a charge force caused by the charges, a capillary force caused by the moisture droplets, or an electrostatic force caused by the static electricity between the particles and the object, is removed so that the particles may be readily removed from the object, particularly, the hole of the object. Therefore, the particles on the inner walls of the hole in the object may be readily removed from the object.

### BRIEF DESCRIPTION OF THE DRAWINGS

The above and other features and advantages of the invention will become readily apparent by reference to the following detailed description when considered in conjunction with the accompanying drawings wherein:

FIG. 1 is a block diagram illustrating an apparatus for removing particles on an object in accordance with a first example embodiment of the present invention;

FIG. 2 is a cross-sectional view illustrating a substrate having holes;

FIG. 3 is a flow chart illustrating a method of removing particles on an object using the apparatus in FIG. 1 in accordance with a second example embodiment of the present invention;

FIG. 4 is a flow chart illustrating a method of removing particles on an object using the apparatus in FIG. 1 in accordance with a third example embodiment of the present invention;

FIG. 5 is a flow chart illustrating a method of removing particles on an object using the apparatus in FIG. 1 in accordance with a fourth example embodiment of the present invention;

FIG. 6 is a block diagram illustrating an apparatus for measuring particles on an object in accordance with a fifth example embodiment of the present invention; and

FIG. 7 is a flow chart illustrating a method of measuring particles on an object using the apparatus in FIG. 3 in accordance with a sixth example embodiment of the present invention.

### DESCRIPTION OF THE EMBODIMENTS

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “includes” and/or “including” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Apparatus for Removing Particles on an Object

Embodiment 1

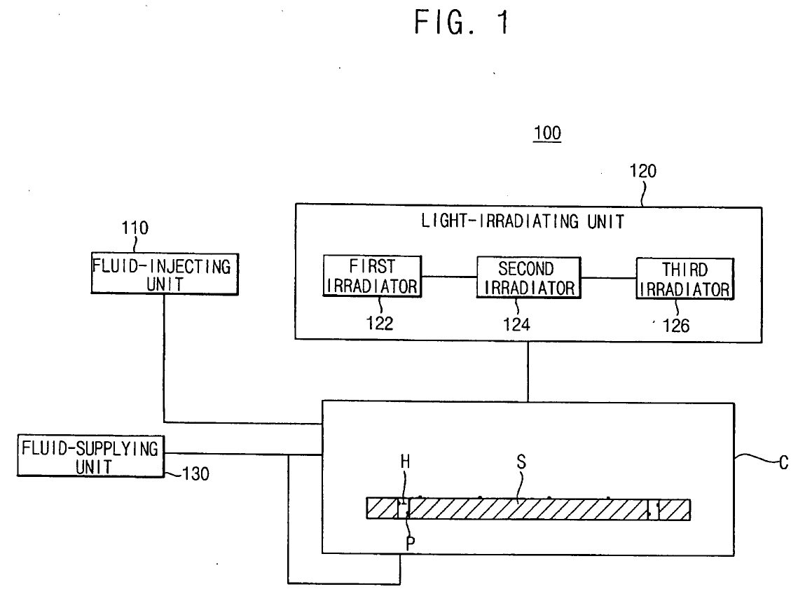

FIG. 1 is a block diagram illustrating an apparatus for removing particles on an object in accordance with a first example embodiment of the present invention.

Referring to FIG. 1, an apparatus 100 for removing particles on an object of this example embodiment includes a measurement chamber C into which an object S is loaded, a fluid-injecting unit 110 for injecting a fluid into the measurement chamber C, a light-irradiating unit 120 and a fluid-supplying unit 130.

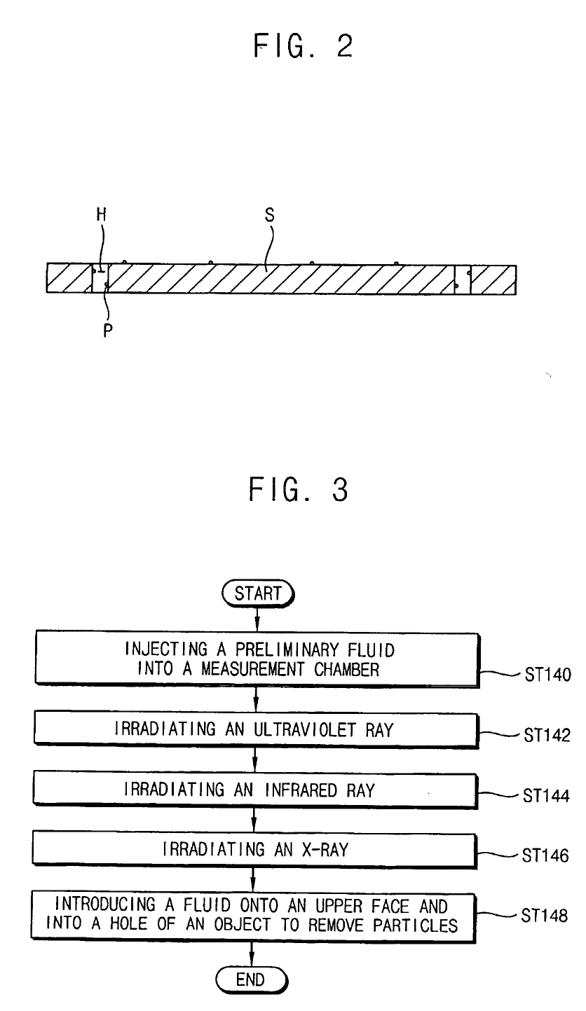

The fluid-injecting unit 110 injects filtered clean air onto the object S on which the particles P are stuck, such as a substrate for a semiconductor device or a flat display device, to remove foreign substances in FIG. 2, such as moisture droplets, in the measurement chamber C. That is, to accurately measure the number of the particles P on the object S, the clean air injected from the fluid-injecting unit 110 blocks inflows of contaminants into the measurement chamber C, to control an environment in the measurement chamber C in advance. Here, as shown in FIG. 2, holes H are formed through the substrate S. Further, the particles P are stuck on inner walls of the holes H as well as an upper face of the substrate S.

The light-irradiating unit 120 removes an adhesion force, such as a charge force, a moisture force, static electricity, etc., between the particles and the object. The light-irradiating unit 120 includes a first irradiator 122, a second irradiator 124, a third irradiator 126 or a combination thereof.

The first irradiator 122 irradiates a first light onto the object to remove charges on a surface of the object S. In this example embodiment, the first light may have a wavelength of about 100 nm to about 400 nm. An example of the first light having the above-mentioned wavelength may include an ultraviolet (UV) ray.

The second irradiator 124 irradiates a second light to an upper face and the hole H of the object S to remove moisture droplets between the particles P and the upper face of the object S, and the particles P and the inner walls of the hole H, and remaining particles that are not removed by the fluid. That is, since the moisture droplets between the particles P and the object S may generate a capillary force, the second light removes the moisture droplets so that the capillary force between the particles P and the object S is removed. In this example embodiment, the second light may have a wavelength of about 0.75 μm to about 1 mm. An example of the second light having the above-mentioned wavelength may include an infrared (IR) ray.

The third irradiator 126 irradiates a third light to the upper face and the hole H of the object to remove static electricity between the object S and the particles P. In this example embodiment, the third light may have a wavelength of about 0.01 Å to about 10 Å. An example of the third light having the above-mentioned wavelength may include an X-ray.

The first to third lights irradiated from the first to third irradiators 122, 124 and 126, respectively, remove the adhesion force between the particles P and the object S. As a result, the particles P may simply rest on the upper face of the object S, and not be adhered on the upper face of the object S.

The fluid-supplying unit 130 introduces the fluid onto the upper face and into the hole H to remove the particles P from the upper face of the object S and the inner walls of the hole H. To introduce the fluid onto the upper face and into the hole H of the object S, the fluid-supplying unit 130 is connected to an upper sidewall and a bottom face of the measurement chamber C to supply the fluid to the upper face and a bottom face of the object S. Here, the fluid supplied from the fluid-supplying unit 130 may be substantially the same as that injected from the fluid-injecting unit 110. Since the particles P simply rest on the upper face of the object S, the fluid supplied from the fluid-supplying unit 130 may readily remove the particles P from the upper face of the object S and the inner walls of the hole H. In this example embodiment, examples of the fluid may include nitrogen gas, argon gas, clean air, deionized water, a soluble or insoluble detergent, etc., having a density of no less than about 99.999%. Further, examples of the detergent may include an acid material, a basic material, a surfactant, etc. Examples of the acid material may include fluoric acid, chloric acid, nitric acid, hydrogen peroxide, etc. Examples of the basic material may include sodium hydroxide, ammonia, etc.

Alternatively, the light-irradiating unit 120 may irradiate the light to the fluid supplied from the fluid-supplying unit 130 as well as the object S. The fluid irradiated by the light is injected onto the upper face of the object S and the inner walls of the holes S to remove the adhesion force between the particles P and the upper face of the object S, and the particles P and the inner walls of the holes P, and simultaneously remove the particles from the upper face of the object S and the inner walls of the holes H.

Method of Removing Particles on an Object

Embodiment 2

FIG. 3 is a flow chart illustrating a method of removing particles on an object using the apparatus in FIG. 1 in accordance with a second example embodiment of the present invention.

Referring to FIGS. 1 and 3, in step ST140, the fluid-injecting unit 110 injects a preliminary fluid such as the clean fluid into the measurement chamber C to remove foreign substances, such as the moisture droplets in the measurement chamber C. The fluid injected from the fluid-injecting unit 110 serves as to provide the measurement chamber C with a desired environment. After the desired environment is formed in the measurement chamber C, the object S is loaded into the measurement chamber C.

In step ST142, the first irradiator 122 irradiates the first light having a wavelength of about 100 nm to about 400 nm to the upper face and the holes H of the object S to remove charges on the upper face of the object S and the inner walls of the holes H.

In step ST144, the second irradiator 124 irradiates the second light having a wavelength of about 0.75 μm to about 1 mm to the upper face and the holes H of the object S to remove the moisture droplets between the upper face of the object S and the particles, and the inner walls of the holes H and the particles P, thereby removing the capillary force between the upper face of the object S and the particles P, and the inner walls of the holes H and the particles P.

In step ST146, the third irradiator 126 irradiates the third light having a wavelength of about 0.01 Å to about 10 Å to the upper face and the holes H of the object S to remove the static electricity between the upper face of the object S and the particles P, and the inner walls of the holes H and the particles P.

Here, the first, second and third lights remove the adhesion force, such as the charge force, the capillary force, or the static electricity between the upper face of the object S and the particles P, and the inner walls of the holes H and the particles P. Thus, since the adhesion force does not exist between the particles and the upper face of the object S, and the inner walls of the holes H and the particles P, the particles P may simply rest on the upper face of the object S and the inner walls of the holes H.

In step ST148, the fluid-supplying unit 130 injects the fluid such as the nitrogen gas, the argon gas, the clean air, the deionized water, the detergent, etc., having a high density to remove the particles P from the upper face of the object S and the inner walls of the holes H, thereby removing the particles P from the object S. Here, as described above, since the particles P merely rest on the upper face of the object S and the inner walls of the holes H, the injected fluid may readily remove the particles P from the upper face of the object S and the inner walls of the holes H.

According to this example embodiment, the adhesion force between the upper face of the object and the particles, and the inner walls of the holes and the particles may be removed using the first, second and third lights. Therefore, the particles may be readily removed from the object so that efficiency for removing the particles may be remarkably improved.

Embodiment 3

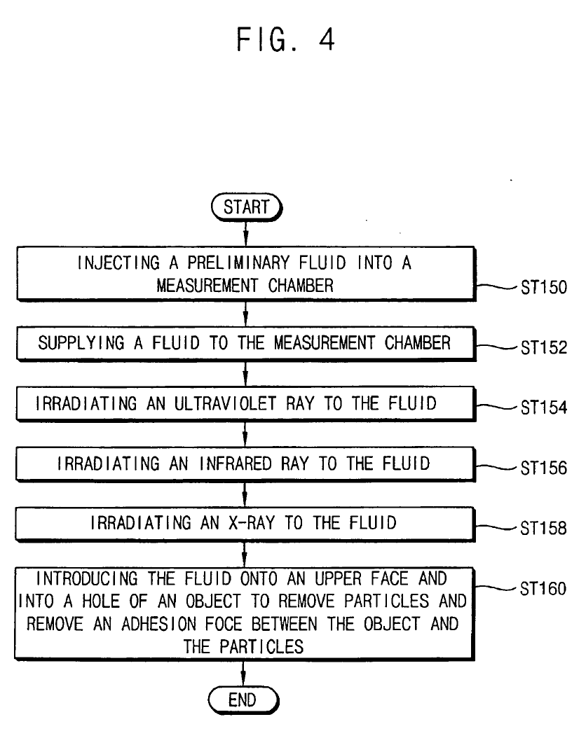

FIG. 4 is a flow chart illustrating a method of removing particles on an object using the apparatus in FIG. 1 in accordance with a third example embodiment of the present invention.

Referring to FIGS. 1 and 4, in step ST150, the fluid-injecting unit 110 injects the clean fluid into the measurement chamber C to remove foreign substances, such as the moisture droplets in the measurement chamber C.

In step ST152, the fluid-supplying unit 130 supplies the fluid to the object S.

In step ST154, the first irradiator 122 irradiates the UV ray having a wavelength of about 100 nm to about 400 nm to the fluid.

In step ST156, the second irradiator 124 irradiates the IR ray having a wavelength of about 0.75 μm to about 1 mm to the fluid.

In step ST158, the third irradiator 126 irradiates the X-ray having a wavelength of about 0.01 Å to about 10 Å to the fluid.

Alternatively, the UV ray, the IR ray and the X-ray may be simultaneously irradiated to the fluid.

In step ST160, the fluid to which the UV ray, the IR ray and the X-ray are irradiated is applied to the upper face and the holes H of the object S to remove the adhesion force between the object S and the particles P, and simultaneously remove the particles P from the upper face of the object S and the inner walls of the holes H, thereby removing the particles P from the object S.

According to this example embodiment, the fluid to which the first to third lights are irradiated may remove the adhesion force between the upper face of the object and the particles, and the inner walls of the holes and the particles, and may simultaneously remove the particles from the upper face of the object and the inner walls of the holes.

Embodiment 4

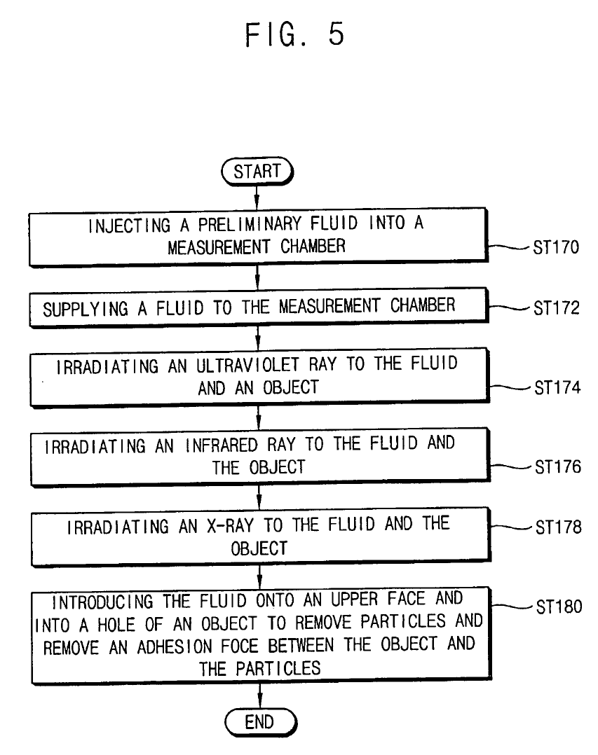

FIG. 5 is a flow chart illustrating a method of removing particles on an object using the apparatus in FIG. 1 in accordance with a fourth example embodiment of the present invention.

Referring to FIGS. 1 and 5, in step ST170, the fluid-injecting unit 110 injects the clean fluid into the measurement chamber C to remove foreign substances, such as the moisture droplets in the measurement chamber C.

In step ST172, the fluid-supplying unit 130 supplies the fluid to the object S.

In step ST174, the first irradiator 122 irradiates the UV ray having a wavelength of about 100 nm to about 400 nm to the fluid and the object S.

In step ST176, the second irradiator 124 irradiates the IR ray having a wavelength of about 0.75 μm to about 1 mm to the fluid and the object S.

In step ST178, the third irradiator 126 irradiates the X-ray having a wavelength of about 0.01 Å to about 10 Å to the fluid and the object S.

Alternatively, the UV ray, the IR ray and the X-ray may be simultaneously irradiated to the fluid and the object S.

In step ST180, the fluid to which the UV ray, the IR ray and the X-ray are irradiated is applied to the upper face and the holes H of the object S to remove the adhesion force between the object S and the particles P and simultaneously remove the particles P from the upper face of the object S and the inner walls of the holes H, thereby removing the particles P from the object S. Further, the first to third lights directly irradiated to the object S may readily remove the particles P from the upper face of the object S and the inner walls of the holes H.

According to this example embodiment, since the first to third lights are irradiated to the fluid as well as the object, the particles on the upper face of the object and the inner walls of the holes may be readily removed.

Apparatus for Measuring Particles on an Object

Embodiment 5

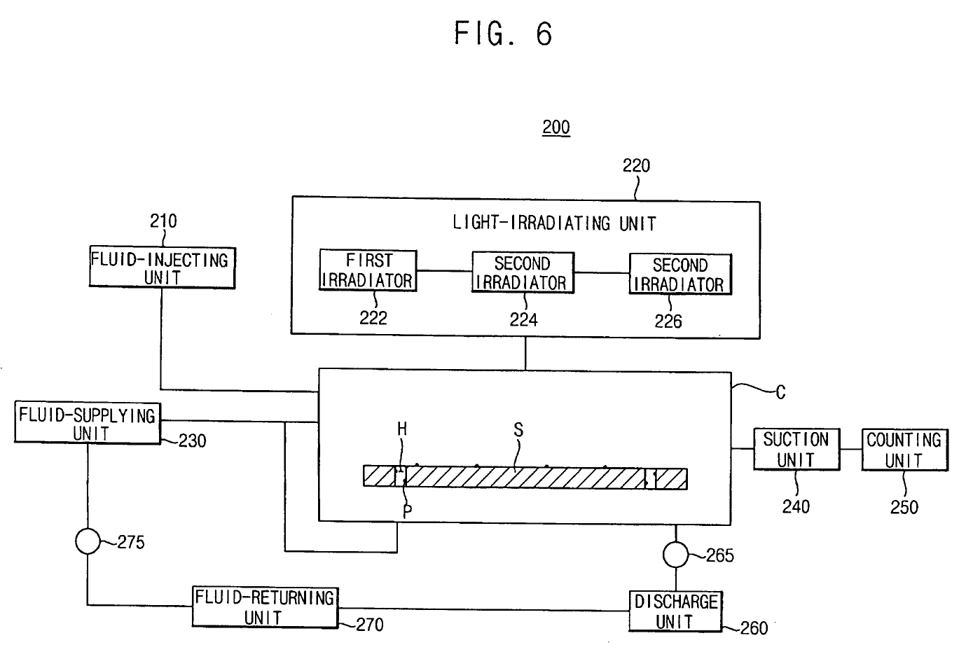

FIG. 6 is a block diagram illustrating an apparatus for measuring particles on an upper face of an object and inner walls of holes in accordance with a fifth example embodiment of the present invention.

Referring to FIG. 6, an apparatus 200 for measuring particles on an object of this example embodiment includes a measurement chamber C into which an object S is loaded, a fluid-injecting unit 210, a light-irradiating unit 220, a fluid-supplying unit 230, a suction unit 240, a counting unit 250, a discharge unit 260 and a fluid-returning unit 270.

Here, the fluid-injecting unit 210, the light-irradiating unit 220, and the fluid-supplying unit 230 are substantially the same as the fluid-injecting unit 110, the light-irradiating unit 120, and the fluid-supplying unit 130 in Embodiment 1, respectively. Thus, any further illustrations with respect to the fluid-injecting unit 210, the light-irradiating unit 220, and the fluid-supplying unit 230 are omitted herein for brevity.

The suction unit 240 sucks the particles P removed from the upper face of the object S and the inner walls of the holes H by the fluid-supplying unit 230 into the counting unit 250. In this example embodiment, the suction unit 240 may include a vacuum pump for providing a space over the object and the holes H with vacuum.

A first flowmeter (not shown) measures a first flow rate of a first fluid introduced to the object S. Further, a second flowmeter (not shown) measures a second flow rate of a second fluid sucked into the counting unit 250. The counting unit 250 counts the number of the particles P in the second fluid sucked by the suction unit 240. Further, the counting unit 250 multiplies the number of the particles P by a flow rate of the first fluid with respect to the second fluid to obtain a value. The counting unit 250 then divides the value by a surface area of the object S to obtain the number of the particles P by unit areas of the object S.

Here, the counting unit 250 may include equipment referred to as a smart probe. In addition, a High-Efficiency Particulate Air (HEPA) filter (not shown), a pressure sensor (not shown), a particle detector (not shown), a particle filter (not shown) may be arranged between the counting unit 250 and the suction unit 240.

The discharge unit 260 discharges a fluid, which is not sucked into the counting unit 250, from the apparatus 200. To accurately recognize a flow rate of the discharge fluid, the discharge unit 260 may include a mass flow controller 265, a flow rate-controllable valve, etc. Further, an example of the discharge unit 260 may include a vacuum pump.

The fluid-returning unit 270 is arranged between the discharge unit 260 and the fluid-supplying unit 230 to return the discharge fluid by the discharge unit 260 to the fluid-supplying unit 230. Additionally, to remove foreign substances in the returned fluid, the fluid-returning unit 270 may include a filter 275.

Method of Measuring Particles on an Object

Embodiment 6

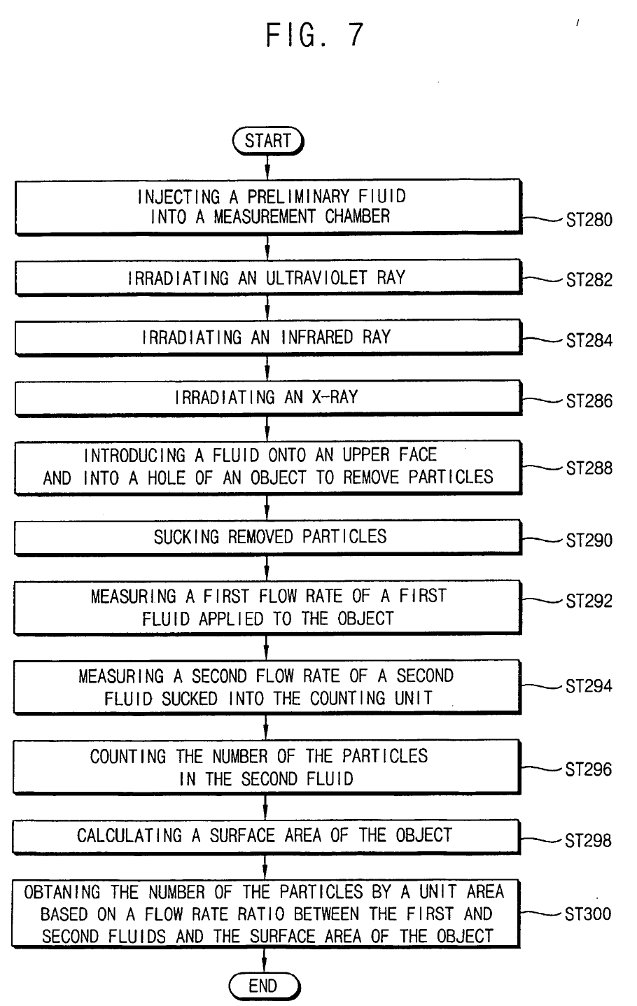

FIG. 7 is a flow chart illustrating a method of measuring particles on an upper face of an object and inner walls of holes using the apparatus in FIG. 6 in accordance with a sixth example embodiment of the present invention.

Referring to FIGS. 6 and 7, in step ST280, the fluid-injecting unit 210 injects the clean fluid into the measurement chamber C to remove foreign substances, such as the moisture droplets in the measurement chamber C. The object S is then loaded into the measurement chamber C.

In step ST282, the first irradiator 222 irradiates the first light having a wavelength of about 100 nm to about 400 nm to the upper face and the holes H of the object S to remove charges on the upper face of the object S and the inner walls of the holes H.

In step ST284, the second irradiator 224 irradiates the second light having a wavelength of about 0.75 μm to about 1 mm to the upper face and the holes H of the object S to remove the moisture droplets between the upper face of the object S and the particles, and the inner walls of the holes H and the particles P, thereby removing the capillary force between the upper face of the object S and the particles P, and the inner walls of the holes H and the particles P.

In step ST286, the third irradiator 226 irradiates the third light having a wavelength of about 0.01 Å to about 10 Å to the upper face and the holes H of the object S to remove the static electricity between the upper face of the object S and the particles P, and the inner walls of the holes H and the particles P.

In step ST288, the fluid-supplying unit 230 injects the fluid such as the nitrogen gas, the argon gas, the clean air, the deionized water, the detergent, etc., having a high density to remove the particles P from the upper face of the object S and the inner walls of the holes H. Alternatively, the first to third lights may be irradiated to the fluid supplied from the fluid-supplying unit 230. Further, the first to third lights may be simultaneously irradiated to the object S and the fluid.

In step ST290, the suction unit 240 provides the measurement chamber with the vacuum to suck the removed particles into the counting unit 250.

In step ST292, the first flowmeter measures the first flow rate of the first fluid introduced to the object S.

In step ST294, the second flowmeter measures the second flow rate of the second fluid sucked into the counting unit 250.

In step ST296, the counting unit 250 counts the number of the particles P in the second fluid.

In step ST298, the surface area of the object S is calculated. Here, the surface area of the object S includes a surface area of the inner walls of the holes H.

In step ST300, the counting unit 250 multiplies the number of the particles P in the second fluid by the flow rate of the first fluid with respect to the second fluid to obtain the value. The counting unit 250 then divides the value by the surface area of the object S to obtain the number of the particles P by the unit areas of the object S. For example, when the first flow rate of the first fluid is 2 CFM (56.64 L/min), the second flow rate of the second fluid is 1 CFM, the number of the particles P in the second fluid is about 120, and the surface area of the object S is about 30 cm², 8 particles/cm² corresponding to the number of the particles by the unit areas of the object S is obtained by a following calculation of 120×(2/1)/30.

According to this example embodiment, the number of the particles by the unit areas of the object may be accurately obtained.

Here, in this example embodiment, the object includes the substrate for the semiconductor device or the flat display device. However, it is obvious to persons skilled in the art that the object is not restricted to the substrate. That is, the methods and the apparatuses of the present invention may be employed in removing particles from other objects.

According to the present invention, the adhesion forces between the particles and the object, and the particles and the inner walls of the holes may be removed using the lights so that the particles may be readily removed from the object and the inner walls of the holes. As a result, the efficiency for removing the minute particles on the inner walls of the holes may be remarkably improved.

Having described the preferred embodiments of the present invention, it is noted that modifications and variations can be made by persons skilled in the art in light of the above teachings. It is therefore to be understood that changes may be made in the particular embodiment of the present invention disclosed which is within the scope and the spirit of the invention outlined by the appended claims.

What is claimed is:

1. A method of removing particles on an object, comprising:
removing an adhesion force between the object and the particles using a light; and
removing the particles from the object using a fluid.

2. The method of claim 1, before removing the adhesion force, further comprising injecting a preliminary fluid into a space where the object is placed to remove foreign substances in the space.

3. The method of claim 1, wherein removing the adhesion force comprises:
irradiating a first light onto the object to remove charges in the object;
irradiating a second light onto the object to remove moisture droplets between the object and the particles; and
irradiating a third light onto the object to remove static electricity between the object and the particles.

4. The method of claim 3, wherein the first light comprises an ultraviolet ray, the second light comprises an infrared ray, and the third light comprises an X-ray.

5. The method of claim 1, wherein the particles are stuck on inner walls of holes formed through the object, and removing the particles comprises introducing the fluid into the holes.

6. The method of claim 5, wherein the fluid comprises nitrogen gas, argon gas, clean air, deionized water or a detergent.

7. The method of claim 6, wherein the detergent comprises an acid material, a basic material or a surfactant.

8. The method of claim 1, wherein the light is irradiated to the fluid, and the fluid to which the light is irradiated removes the adhesion force and simultaneously removes the particles.

9. A method of measuring particles on an object, comprising:
removing an adhesion force between the object and the particles using a light;
removing the particles from the object using a fluid; and
counting the number of the removed particles.

10. The method of claim 9, wherein the particles are stuck on inner walls of holes formed through the object, and removing the particles comprises introducing the fluid into the holes.

11. The method of claim 10, wherein counting the particles comprises:
measuring a first flow rate of a first fluid introduced to the object;
measuring a second flow rate of a second fluid used for counting the particles;
counting the number of the particles in the second fluid;
calculating a surface area of the object;
obtaining a value by multiplying the number of the particles in the second fluid by a flow rate of the first fluid with respect to the second fluid; and
dividing the value by the surface area of the object to obtain the number of the particles by a unit area of the object.

12. The method of claim 9, wherein the light is irradiated to the fluid, and the fluid to which the light is irradiated removes the adhesion force and simultaneously removes the particles.

13. An apparatus for removing particles on an object, comprising:
a light-irradiating unit for irradiating a light onto the object to remove an adhesion force between the object and the particles; and
a fluid-supplying unit for supplying a fluid to the object to remove the particles from the object.

14. The apparatus of claim 13, further comprising a fluid-injecting unit for injecting a preliminary fluid into a space where the object is placed to remove foreign substances in the space.

15. The apparatus of claim 13, wherein the light-irradiating unit comprises:
a first irradiator for irradiating a first light onto the object to remove charges in the object;
a second irradiator for irradiating a second light onto the object to remove moisture droplets between the object and the particles; and
a third irradiator for irradiating a third light onto the object to remove static electricity between the object and the particles.

16. The apparatus of claim 15, wherein the first light comprises an ultraviolet ray, the second light comprises an infrared ray, and the third light comprises an X-ray.

17. The apparatus of claim 13, wherein the light-irradiating unit irradiates the light to the fluid to remove the adhesion force and simultaneously remove the particles using the light to which the light is irradiated.

18. An apparatus for measuring particles on an object, comprising:
a light-irradiating unit for irradiating a light onto the object to remove an adhesion force between the object and the particles;
a fluid-supplying unit for supplying a fluid to the object to remove the particles from the object;
a counting unit for counting the number of the removed particles; and
a suction unit for sucking the removed particles into the counting unit.

19. The apparatus of claim 18, further comprising a discharge unit for discharging a fluid that is not sucked into the counting unit.

20. The apparatus of claim 19, wherein the discharge unit comprises a mass flow controller.

21. The apparatus of claim 18, further comprising a fluid-returning unit arranged between the discharge unit and the fluid-supplying unit to return the fluid for removing the particles to the fluid-supplying unit.

22. The apparatus of claim 21, wherein the fluid-returning unit comprises a filter for removing foreign substances in the fluid.
